(19) United States
(12) Patent Application Publication
(10) Pub. No.: US 2006/0129113 A1
(43) Pub. Date: Jun. 15, 2006

(54) Eye drop applicator

(76) Inventor: James Merrick, Kerrville, TX (US)

Correspondence Address: Daniel D. Chapman;Jackson Walker, LLP, Suite 2100, 112 E. Pecan, San Antonio, TX, 78205, US

(21) Appl. No.: 11/297,851

(22) Filed: Dec. 8, 2005

Related U.S. Application Data
(60) Provisional application No. 60/634,646, filed on Dec. 9, 2004.

(51) Int. Cl. A61M 35/00

(52) U.S. Cl. 604/294

(57) ABSTRACT

A novel applicator for engagement to an eye drop vial. The eye drop vial would be squeezable and contain a liquid therein that is compatible with the eye. The adapter includes a neck having a multiplicity of inner diameters adapted to a multiplicity of eye drop vials and a cup. The cup is semi-spherical shaped and has a rim that is collapsible between a closed position, which would seal off the vial, and an open position which would allow application of liquid from the vial to the eye of a user.

This is a utility patent application that claims priority from U.S. Provisional Patent Application Ser. No. 60/634,646, filed Dec. 9, 2004.

### FIELD OF THE INVENTION

Eye drop applicators, namely and eye drop applicator capable of engaging a flexible, squeezable eye drop vial and moveable between a collapsed or nonuse position to a use or open position.

### BACKGROUND

Eye drops typically come in a vial. The vial typically includes a nozzle having a tip. The vial is inverted and, the body being flexible, squeezed to dispense a drop onto the open eye.

Improved eye drop applicators are also provided such as those set forth in the following patents: U.S. Pat. No. 3,945,381; DES248,448; 5007,905; U.S. Pat. Nos. 5,295,981; and 5,578,019.

The aforementioned patents all have in common some type of an adapter designed to engage the eye drop containing vial to help assist the user in the application of the drop from the vial to the eye.

Numerous devices have been proposed in which a person can apply eye drops or medicine into the eye without assistance. Such devices include the well-known eyewash cup device where an eyewash liquid is poured into a cup adapted to fit against the rim of the eye socket and the cup then applied to the eye area. Other devices consist of squeeze bottles with dispensing tips with or without a nose bridge guide arrangement.

The eye drop cup, however, being a separate article, may be lost or misplaced. A squeeze bottle with a dispensing tip with or without guide arrangements may be inherently dangerous in that the narrow dispenser tip can cause damage to the eye if the user causes the tip to make contact with the eye. Further, most squeeze bottles with engagement devices for engaging the perimeter of the eye socket have a mouth, the mouth being opened and capable of receipt of contamination therein.

U.S. Pat. No. 3,945,381 discloses an eye drop dispenser assembly having a squeezable vial of conventional construction for containing an eye drop solution therein. The vial contains an integral neck and threaded portion, the threaded portion for engaging a conventional cap. This patent discloses an eye drop cup which is adapted to be secured to the neck of the conventional dispenser vial. A separate cover is provided to engage the cup which in turn engages the dispensing vial. The cover is designed to simultaneously seal the cup and the container to protect both from bacteria or other contaminants.

U.S. Pat. No. 5,295,981 discloses yet another variation of an eye drop cup for engagement with the threaded portion of a standard flexible eye drop containing vial. The '981 Patent also illustrates the use of a cap for engaging and closing off the open end of the eye cup.

Both the eye cups disclosed in the '981 and '381 patents have generally cylindrical or oval open mouth portions that are designed to be urged against the eye socket. Both of the patents also disclose the use of separate caps for preventing bacteria or other contaminants from obtaining access to the tip of the vial.

It may be appreciated that while the function of the cap—the prevention of contamination obtaining access to the tip of the vial, is laudable, separate caps would be easily misplaced. Thus, Applicant has provided a novel flexible cup with a mouth capable of moving or being moved between a closed position in which the mouth of the cup is substantially closed and an open position in which the mouth of the cup is open in a ready, use or dispensing position. That is to say, Applicant provides a novel cup in which the cup itself is adapted to close or be closed and seal the interior of the cup and the tip of the applicator from contamination.

### OBJECTS OF THE INVENTION

Therefore, it is the object of Applicant's present invention to provide a coverless eye drop applicator vial attachment that can nonetheless prevent contamination of the tip of the vial.

It is a further object of Applicant's present invention to provide a simple, easy to manufacture and easy to use eye drop applicator that will act as a guide to the user in directing eye drops from the tip of the applicator to the eye.

It is a further object of the present invention to provide an eye drop applicator with an eye drop guide, which can be operated with one hand.

It is a further object of Applicant's present invention to provide a method for using a novel eye drop applicator in the first minutes of the day, as the user begins to wake up, when the eye is the driest.

It is a further object of the present invention to provide an applicator that can be used with a single hand and without the need for sight, allowing, therefore, the application of eye drops to the eyes while they are closed and in the dark.

It is another object of the present invention to provide a mass produced adapter for an eye drop vial that is capable of fitting any sized preferably squeezable eye drop vial.

It is a further object of the present invention to provide an eye drop applicator which is capable of moistening eye membranes which have dried throughout the night and help wash the eyes of sleep.

It is yet another object of the present invention to provide an eye drop applicator that will provide the user with clearer eyesight before leaving bed in the morning, especially in darkness.

It is yet a further object of Applicant's present invention to provide for a novel eye drop applicator with an effective, safe applicator guide which is placed around the desired eye for accurate instillation of the eyes while the eyes remain closed.

It is yet another object of the present invention to provide a novel combination of Applicant's eye drop applicator and a liquid for use therein, which liquid provides for rapid washout of an eye including an eye of contact lens users.

### SUMMARY OF THE INVENTION

This and other objects are provided in an eye drop applicator including a standard squeezable eye drop vial containing eyewash lubricant liquid therein, which vial is provided with a cup guide that occludes a bowl and a neck.

These and other objects are achieved in providing a novel eye cup guide with a bowl and neck, the bowl flexible and adapted to move, in response to finger pressure by the user, between a closed position where the perimeter of the bowl is substantially sealed and an open or use position where the perimeter of the bowl assumes an open shape that is designed to engage the socket of an eye.

These and other objects are provided in providing a cup guide having a bowl and a neck, the neck having an interior perimeter defining a multiplicity of diameters, the neck being capable therefore of adapting to a variety of standard sized eye drop vials.

These and other objects are provided in a method of using Applicant's novel eye drop applicator for the application of eye drops to a closed eye in the dark using only a single hand.

These and other objects are provided in the use of Applicant's novel eye drop applicator in conjunction with an eye drop formula found effective for use in washing and lubricating the eyes after a nights sleep.

### BRIEF DESCRIPTION OF THE DRAWINGS

FIG. 1 is a perspective view of Applicant's novel eye drop applicator illustrating the use of the cup guide having a bowl and a neck for engagement with a squeezable eye drop vial.

FIG. 2 is a top elevational view of the cup guide in a closed position.

FIG. 3 is a side elevational view of the cup guide in a closed position illustrating the segmented neck and the mouth in a closed position.

FIG. 4 is a side elevational view as set forth in FIG. 3 with the cup guide rotated 90 degrees.

FIG. 5A is a side elevational cutaway view of a portion of the cup guide including the rim thereof showing the ring embedded within the rim defining the mouth.

FIG. 5B is a side elevational view of the cup guide in a partially open position.

FIG. 5C is a side elevational view, cutaway, of the mouth in a fully open position illustrating the ring embedded in the rim.

FIG. 6A is a top elevational view of the cup guide in an open position.

FIG. 6B is a side elevational view, partially cut away, to show a cross-sectional view of the manner in which the segmented neck may be adapted to fit an eye drop vial having a threaded base. This illustration demonstrates adaptability towards a conformation of shape when presented with a smaller eye drop vial type simply by the elimination of the two lower rings of the neck of the applicator. The material of the neck is stretchable to some degree so as to make a snug fit on the bottle.

FIG. 6C illustrates the use of Applicant's novel cup guide for engagement with an eye drop vial having a threaded base that is larger in diameter than the threaded base of the eye drop vial illustrated in FIG. 6B. This is accomplished by inclusion of all three rings of the neck of the applicator.

FIG. 7A illustrates a side elevational view of a user illustrating the one hand application of Applicant's novel eye drop applicator to apply drops to the user's eye.

FIG. 7B is the same as FIG. 7A with the exception of the user turning the vial to an inverted position and squeezing to emit a drop of liquid from the vial, illustrating the manner in which the rim of the bowl engages the socket of the eye.

FIG. 7 is a top elevational view of the ring for use in conjunction with the bowl of Applicant's novel cup guide, the top and bottom elevational views being the same.

FIG. 8 is a side elevational view of the ring of Applicant's novel cup guide, the left and right side elevational views being the same.

FIG. 9 is a side elevational view of the ring of Applicant's present invention showing the first and second waist portions to the left and to the right, wherein the view set forth in FIG. 8 shows only one waist portion.

FIG. 10 is a perspective view of the ring apart from the cup and shows the manner in which the ring is positioned when the mouth is open.

FIGS. 11A and 11B illustrate a top elevational and side perspective view of an alternate preferred embodiment of Applicant's present invention, which embodiment provides an un-tensioned ring member for movement between an opened and closed position.

FIG. 13A illustrates the un-tensioned ring member part from the eye cup, in perspective view.

FIG. 13B illustrates a top elevational view of the rim of the eye cup of the alternate preferred embodiment set forth in FIGS. 11A and 11B, showing the un-tensioned ring member imbedded therein.

FIGS. 13C and 13D are cutaway cross-sectional views of the stabilizing members and position holding members of the embodiment set forth in FIGS. 11A, 11B, and 12A.

FIG. 14 illustrates a second alternate preferred embodiment of Applicant's present invention in cutaway perspective view illustrating another un-tensioned ring member.

FIGS. 15A and 15B illustrate a third alternate preferred embodiment of Applicant's present invention which includes an un-tensioned perimeter, but with no member at all imbedded therein, simply a thicker rubber or polymer perimeter, normally closed, but which has memory built therein and which will remain in an opened position for a period of time, typically 30 seconds to 2 minutes, then slowly return back to its original closed position.

### DETAILED DESCRIPTION OF PREFERRED EMBODIMENTS

The figures illustrate several embodiments Applicant's novel eye drop applicator (10) including a squeezable, standard off the shelf eye drop vial (12) containing a liquid eyewash or eye lubricant (14) therein. The figures illustrate a cup guide (16) for engagement with the eye drop vial (12).

FIGS. 1 through 6C, illustrate the nature of Applicant's cup guide (16), seen to be comprised of a flexible material such as a rubber, latex or a substance preferably comprised of a copolymer of ethylene and propylene. This combination is substantially nontoxic and biologically inert. Advantages provided by the elastomeric nature of this material provides not only adaptability to fit various sized vials but also provides safety for the user.

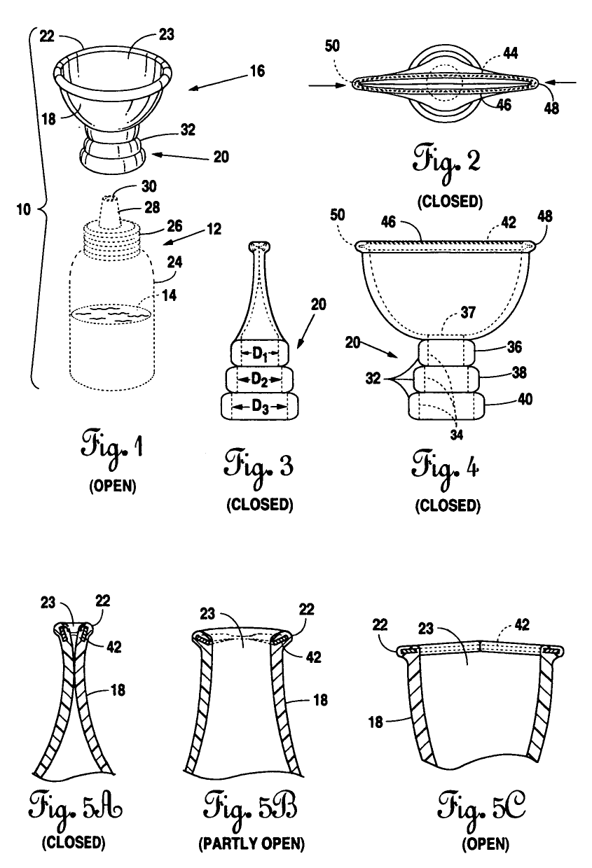

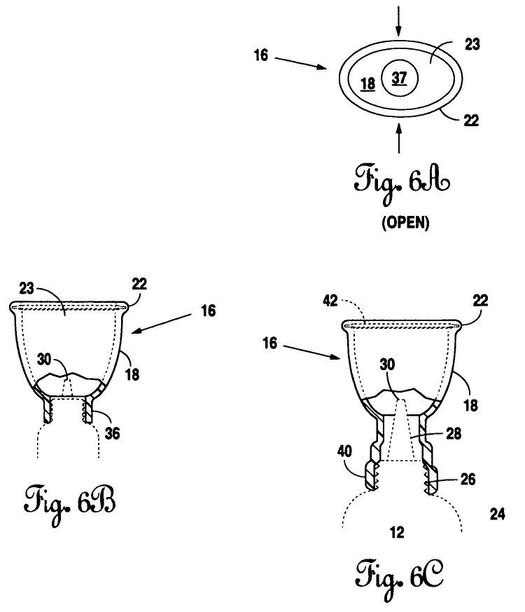

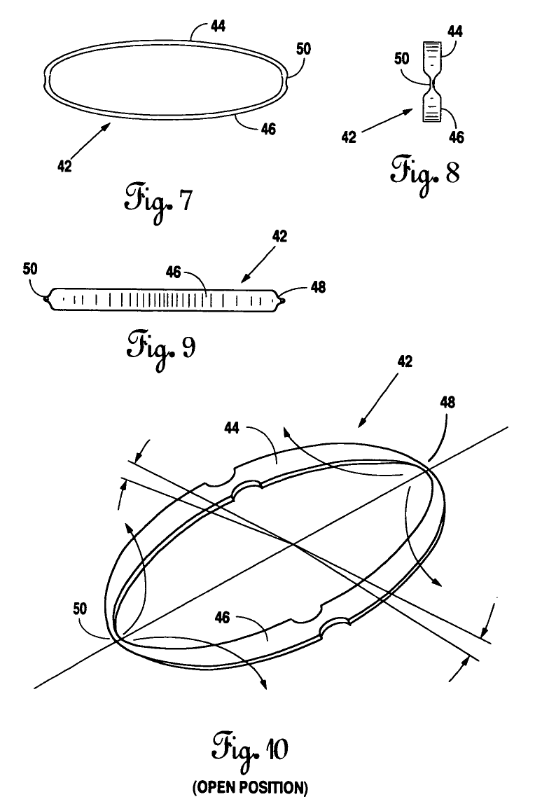

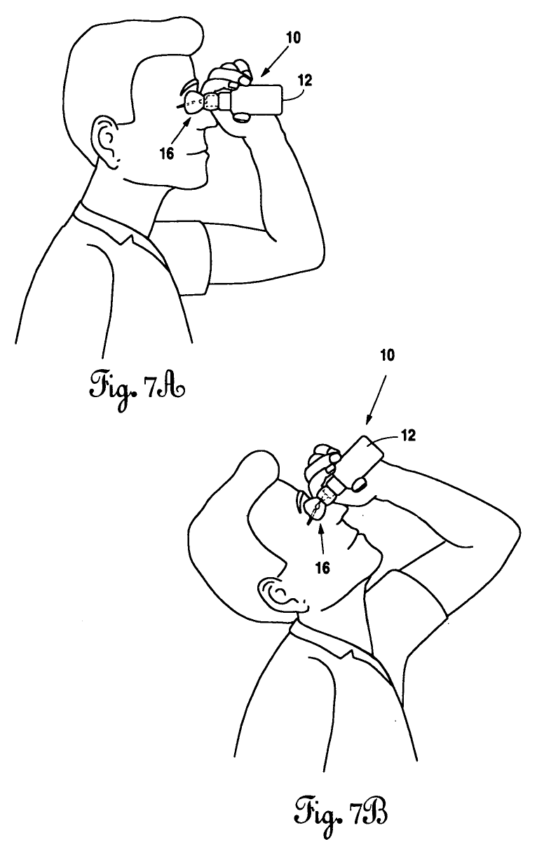

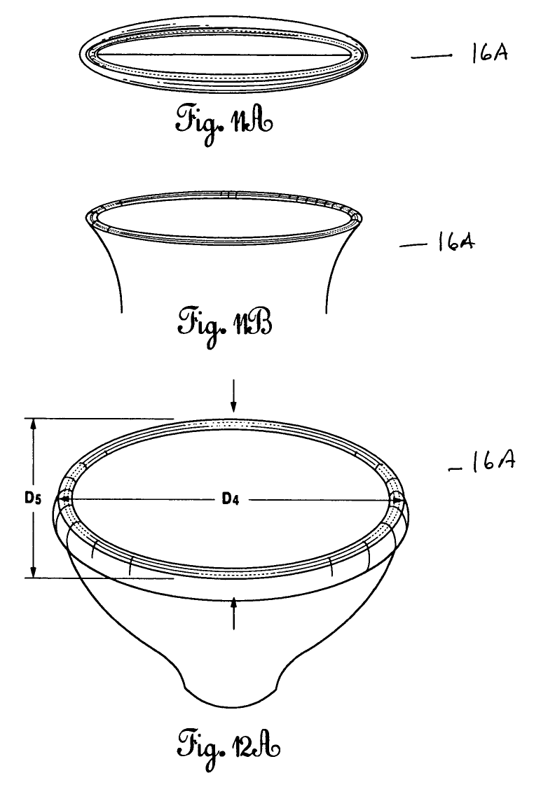

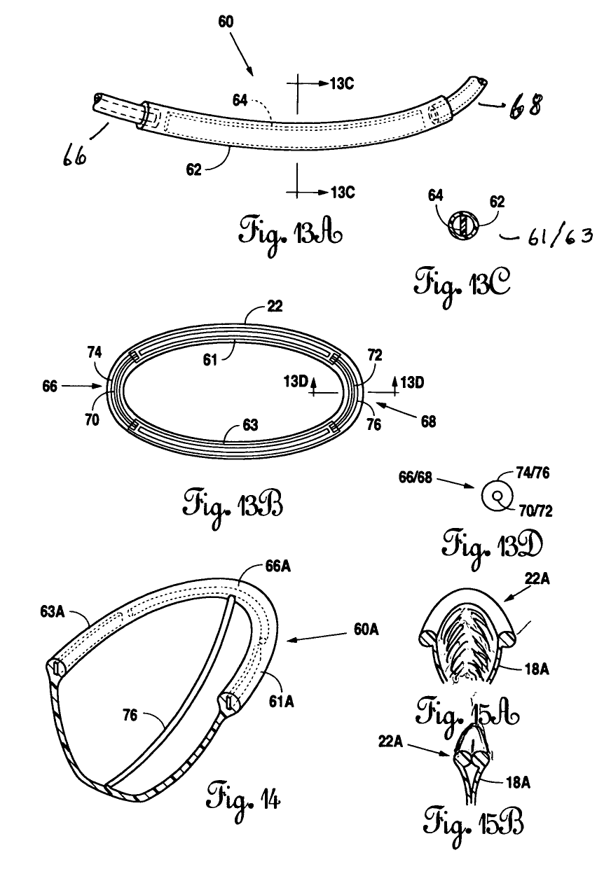

Furthermore, it is seen that Applicant's novel cup guide includes a bowl (18) having a rim (22) defining a mouth (23) (which may assume an open or closed position as set forth in more detail below) and, opposite the mouth, a segmented neck (20) open to the base of the bowl. Bowl (18) is generally, in an open position, semi-spherical or semi-ovoid as seen in FIGS. 5C, 6A, 12A, 14, and 15A, when in use, and in a closed position, when not in use, as seen in FIGS. 2, 3, 5A, 11A, and 15B, has the shape of a narrow closed oval. The open position is shaped to engage the eye socket and the closed position substantially seals the bowl of the cup guide.

Turning now back to FIG. 1, it is seen that vial (12) includes a typical squeezable vial body (24) for receipt of a liquid (14) therein. Typical vial (12) body sizes range anywhere from a capacity of 2.5 ml to 60 ml. Further, vial body is seen to, typically, include a threaded base (26) for receipt of a cap (not shown) thereon. Threaded base (26) terminates in an elongated nozzle (28) having a tip (30), the nozzle and tip for directing the liquid there through. Threaded base (26) is typically cylindrical and typically defines one of the following three diameters: D1=about 11 mm, D2=about 14 mm, D3=about 16 mm.

With reference to FIGS. 1 through 6C, it is seen that Applicant's novel cup guide (16) includes a flexible segmented neck (20) defining a neck outer surface (32) and a neck inner surface (34). Moreover, it is seen that segmented neck is segmented at the outer surface thereof to define, in a typical embodiment, three neck portions: first neck portion (36), second neck portion (38) and third neck portion (40). First neck portion (36) defines an inner diameter D1, second neck portion defines an inner diameter D2 and third neck portion defines an inner diameter D3, diameter D1 being less than diameter D2 which is in turn less than diameter D3. The use of Applicant's novel segmented neck, segmenting the inner diameters into two or more different interior diameters provides a novel approach for adapting Applicant's novel cup guide to any one of two or more standard threaded bases. For example, diameter D1 may equal approximately 11 mm, diameter D2 may equal approximately 14 mm, and diameter D3 may equal approximately 16 mm. Further, material of Applicant's novel cup guide is sufficiently soft and pliable to be cut between portion (36) and (38) such that the neck of cup fits on to a vial (12) having a diameter D1 or slightly greater than the inner diameter D1. The segmented neck (20) may be left uncut such that it may be used with a vial (12) having a threaded base of a diameter slightly larger than D3. In such a case, third neck portion will, being slightly stretchable or pliable, fit snugly over the threads of the threaded base and will maintain the cup guide (16) in the position illustrated in FIG. 6C. Moreover, when the cup guide is engaged to the vial and the mouth in the closed position, it is seen that little or no dust or contaminants will enter the interior of the cup and contaminate the same or tip (30). Further, it is seen with reference to FIGS. 5C, 6, 7A and 7B that the bowl, when in an open position, defines a mouth that will fit snugly against the eye socket and provide a guide for eye drops from within the squeezable vial to, when the vial in an inverted or substantially inverted position as set forth in FIG. 7B, contact the eye.

Turning now with reference to the Figures, especially FIGS. 5A through 5C and FIGS. 7 through 10, it is seen that Applicant's bowl (18) includes a rim (22), the rim (22) having, embedded therein, a ring (42) having the unique shape set forth in FIGS. 7 through 10. Ring (42) is capable of assuming one of two positions, an open or a closed position. The closed position is illustrated in FIGS. 2, 3, 4 and 5A and the open position is illustrated in FIGS. 1, 5C, 6A, 7A and 7B. The ring in an open position is illustrated in FIGS. 7 through 10. Ring (42) has the unique geometry as set forth in FIGS. 7 through 10 and, when embedded in rim (22), provides for maintenance of the bowl in the closed position set forth in FIG. 2.

Ring (42) is comprised of a first tabular section (44) and a second tabular section (46). The term tabular refers to the cross-sectional shape of these two sections which are substantially identical in shape and which, in profile, define when connected at waist portion (48) and (50), the shape of the open mouth as set forth in FIGS. 6A and 7, FIG. 6A having the ring embedded in the rim and FIG. 7 the ring apart from the rest of the bowl.

In FIG. 8, it is seen that waist portions (48/50) represent a narrowing or pinched portion, see also FIGS. 7, 9 and 10. The geometry of this shape allows the ring, and the rim in which it is embedded to, assume either a stable open or a stable closed position. When assuming the stable open position, if force were to be exerted as set forth by the arrows in FIG. 6A, the ring would collapse or “pop” to the closed position as set forth in FIG. 2. On the other hand, if the ring is in the closed position as set forth in FIG. 2 and force is exerted in the direction of the arrows by for example, the fingers of the user, the ring will “pop” to the open position. FIG. 5B is a partly open/partly close (unstable) position of the ring embedded in the rim. Note how the ring is adapted to pivot within the rim, see for example FIG. 5A close position and FIG. 5C open position.

Ring (42) may be made from metal, rubber, plastic, combination of soft latex coating or hard rubber, or other suitable material. FIG. 7A and FIG. 7B illustrate the use of the eye drop applicator (10). The cap (not shown) of the eye drop applicator is typically discarded and the neck is sized, as by trimming if necessary, to snugly engage threaded base (26) of the vial. When not in use, the mouth is urged to the closed position or is naturally resting in the closed position. Typically, Applicant's eye drop applicator (10) is placed by the bedside at night while snugly engaged to the threaded base (26) of the vial. In the morning, when the alarm goes off, the eyes are typically dry. The user may simply reach over, without opening the eyes, and grasp the vial body (24) and, with his index finger and thumb, gently squeeze at the points representing the tips of the closed mouth as seen in FIG. 2 to allow the rim to spread out to an open position as seen in FIG. 6A. At this point, with, typically, the eyes still closed, the user may or may not rest his hand against his or her nose or any other comfortable location on the face and snugly receive, by feel, the rim against the eye socket. The soft flexible rim of the applicator provides the user a safe mechanism to accurately position the exact desired location he or she wishes the drop or drops to fall. The desired target being either the crease between the eyelashes or in the corner of each closed eye. If the head is not upturn, the applicant can upturn the head and invert the vial as seen in FIG. 7B however the most common application position is probably in the supine position. Gently squeezing the squeezable vial will allow a drop or more of liquid to fall gently within the creases of each closed eyelid. The user may then open his eyes and allow the wash to enter and contact the eyeball. The unit can then be placed back down on a support surface for preparation of arising from bed. Gently squeezing the rim in the directions indicated by the arrows as set forth in FIG. 6A will “pop” the mouth of the close position. Gently squeezing the rim in the directions indicated by the arrows as set forth in FIG. 6A, will close the mouth into a closed or sealed position.

Turning back to FIG. 7A, and “mentally” rotating it 90°, one can imagine the user laying on his back on a bed, with the longitudinal axis of the unit perpendicular to the plane of the body.

It is to be understood that the vial may be used with any eye compatible liquid therein, but in a preferred embodiment, the liquid represents substantially the following: sterile water 99.0%; sodium chloride 0.9%; EDTA 0.02%; and benzalkonium chloride 0.01%. This is a buffered solution that approximates, in part, the salt content of the eye, contains a bacteria inhibitor and a stabilizer. This is an example only, the nature of the liquid dispensed is not part of the invention.

It is to be further understood that the eye drop applicator may be used to apply any liquid, either a contact lens solution, an eyewash, or any other eye medication. It is to be further understood that the applicator may be used without the novel neck or the novel neck may be used without the novel automatic closing mouth.

The embodiment of Applicant's novel eye drop applicator may be understood with reference to FIGS. 6A, 6B and 6C. In the first embodiment, ring (42) is uniquely tensioned to “pop” between an open and closed position. Un-tensioned ring member (60) of the second embodiment 16A, as illustrated in FIGS. 11A-14, however, is constructed such that applying forces indicated in FIG. 11A will move the ring member to an open position (FIG. 12A) (see also FIG. 6A). Un-tensioned ring member (60) will simply stay in the position that the user's fingers place it in. There is no “pop open” or “pop close” action as in the previous embodiment. The un-tensioned ring member (60) includes positioning member (66/68) connected to stabilizing portions (61/63) as set forth in FIGS. 13A, 13B, 13C and 13D.

The overall function of this ring is to take a position that is set by the application of force, when opening from a closed position (see FIG. 11A) (see also FIG. 2) at the ends to an open position as seen in FIG. 12 (see also FIG. 6A), when such position defines, a substantially flat opening approximately slightly larger than an open eye and in a similar overall oval shape. The preferred dimensions of the opening of the cup as set forth in FIG. 12A are preferably, but not necessarily, D4=about 34 mm to 36 mm (major axis) and D5=about 24 mm to 26 mm (minor axis).

Thus, in the second embodiment of Applicant's present invention un-tensioned ring member (60) is adapted to be folded by relatively constant force from the fingers of the user from the position in FIG. 11A to the position in FIG. 12A and, from a position in FIG. 12A by application for force as indicated by the arrows in FIG. 12A back to the close position of FIG. 11A. The difference between the first embodiment illustrated in FIGS. 1-10 and the second embodiment with the un-tensioned ring (60) is in the second embodiment and will not pop open on its own and then pop closed. In the present embodiment, the un-tensioned ring will move as force is applied and stop when force is not applied. Moreover, in Applicant's novel un-tensioned ring (60), the adaptation of the present embodiment provides for the ring to maintain a relatively flat planar position as illustrated in FIG. 12A when force is applied between the close position and the open position. That is, the novel design of Applicant's un-tensioned ring (60) will result in the perimeter maintaining a relatively flat shape when viewed from the side.

Thus, in Applicant's second embodiment illustrated in FIGS. 11A and 12A, the use of a combination of stabilizing members to maintain a relatively flat plane between the open and close position as well as wire metal containing positioning members will provide for relatively flat planar movement between an open and close position.

Positioning or position holding members (66/68) are typically similarly dimensioned and may include wire members (70/72) that may be placed within plastic, rubber or other suitable coating material (74/76) (see FIGS. 13B and 13D). The function of position members is to leave the rim in the desired position when user applied force is released. Typically, positioning members (66/68) are adapted to engage stabilizing members (61/63) in the manner set forth in FIGS. 13A and 13B. Stabilizing members (61/63) typically include a rubber, vinyl or other suitable tubular material (62) which has, encased therein, a plastic band (64) or other flat tabular shaped member that is placed edgewise within the perimeter as best indicated in FIGS. 13A and 13B and positioned as seen in FIG. 13C. By placement of an elongated tabular member such as plastic band (64) or other resilient material in the position set forth in FIGS. 13A and 13B, movement of the perimeter between the close position and the open position will be constrained to a relatively flat plane. This is believed to be due in part to the tabular construction of the plastic band, its resiliency and its confinement within the tubular material (62) in the manner best illustrated in FIG. 13C.

FIG. 14 illustrates a perspective view of yet another alternate embodiment of Applicant's un-tensioned ring member (60A). In the embodiments disclosed in FIG. 14, alternate preferred positioning members include (66A) which is merely a wire member such as 16 to 18 gauge copper wire that is directly embedded within the perimeter of the cup. Stabilizing members (61A/63A) are tabular and are molded or otherwise embedded directly in the material comprising the perimeter of the cup. In FIG. 14; the application of force as indicated in FIG. 11A and the application of force as indicated in FIG. 12A will result in generally planar movement between the open and close position in the same manner as the embodiment illustrated in FIGS. 13A through 13D.

The body of the cup partially constrains the movement of the perimeter between an open and close position. Typically, when moving between a close and an open position, the body of the cup will constrain the ring member from bending outward. Stabilizing members will help constrain the perimeter from “folding” inward. Positioning members, typically being wire rod members will be constrained in their motion by both the effect of the stabilizing members and the integral nature of the body in the perimeter of the cup.

Un-tensioned ring members 60/6A may be simply encased within a molded latex body and rim as illustrated or positioning members could be made of any suitable material that will retain its position when forced into a desired shape. Stabilizing members may be of any configuration that will prevent the perimeter of the cup from moving or folding out of a general planar configuration or otherwise allow a smooth movement between a close position as in FIG. 11 and a open position as illustrated in FIG. 12. An additional feature that may be used is thickened rib (76), which may be simply a thickened rib shape portion of the latex or other material may be provided. Rib (76) will assist in adding stability to the cup, especially when moving between an open and close position.

There are different types of rubber and/or latex materials as they relate to quickness of response from a distorted to an original position. They can be generally categorized as rapid response or delayed response, describing the ability of the material to respond to a distortion from its original position to recovery back to that original position. It is possible then, and provided herein, to make at least the rim of the cup, or the perimeter, entire cup and neck assembly, of a material that has an original undisturbed “memory” position in the closed position. Application of a force to the apex or corners, as set forth herein above, then would allow the cup to move from the closed to the open position, as set forth herein above. The material may be designed to maintain the open position (delayed recovery to the original close position) for a time sufficient, for example 30 seconds to two minutes, such that the user would not have to apply any force at all to “reclose” the cup. The delayed recovery would allow the rim to close at its own speed after the user has placed the unit, for example back on the bedside table, having completed the application of drops to the eyes. This is accomplished by modifying rubber or polymers in ways known in the chemical trade, to apply the appropriate polymerization to the latex, rubber, polymer or other material, to at least the material making up the rim. This third embodiment is illustrated in FIGS. 15A and 15B. FIG. 15A shows an eye cup with body (18A) and rim (22A), in the open position. Typically, the rim (22A) would be made of a polymer with a slow response that would maintain a normally “memory” closed position, as set forth in FIG. 15B. Body (18A) may be made of the same material that rim (22A) is made out of or a different material.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

1. A cup guide to adapt to a squeezable liquid bearing container, the cup guide comprising:
a flexible rim member defining a mouth;
a flexible bowl member, integral with the flexible rim member; and
an elastomeric neck integral with an opening to the bowl, the elastomeric neck further having an opening adapted to receive a dispensing portion of the fluid bearing container.

2. The cup guide of claim 1, wherein the rim is capable of maintaining either a closed position or an opened position and further capable of movement between the opened and closed positions, such movement responsive to pressure applied by the user to the rim.

3. The cup guide of claim 2, wherein the rim includes tension means to drive the rim between an opened and closed position.

4. The cup guide of claim 2, wherein the rim includes means to selectively locate and indefinitely maintain the rim in the opened position, closed position or position intermediate thereto when a user applied force is removed therefrom.

5. The cup guide of claim 2, wherein the rim includes a polymer having an extended memory for maintaining the closed position and for providing a delayed response to movement towards a closed position when user applied force places the rim in the opened position.

6. The cup guide of claim 5, wherein the neck has a multiplicity of inner diameters adapted to fit a multiplicity of liquid bearing container sizes.

7. The cup guide of claim 6, wherein the external shape of the neck is segmented to locate the multiplicity of inner diameters.

8. The cup guide of claim 5, wherein the bowl includes a stiffening rib.

9. The cup guide of claim 6, wherein the inner diameters are about 11 mm., about 14 mm., and about 16 mm.

10. The cup guide of claim 5, wherein the mouth, in an opened position, defines an oval having a major axis between 34 mm and 36 mm and a minor axis between 24 mm and 26 mm.

11. A method of dispensing onto the eye of a user liquid from a squeezable liquid container, the method providing:
a flexible rim member defining a mouth;
a flexible bowl member, integral with the flexible rim member;
an elastomeric neck integral with an opening to the bowl, the elastomeric neck further having an opening adapted to receive the fluid bearing container;
applying a force to the mouth to move the rim towards an opened position;
upturning a bottle;
placing the bottle with the mouth opened adjacent the socket of the eye; and
squeezing the bottle to apply a desired amount of eye drops to the eye.

12. The method of claim 11, further including applying a force to the mouth to move the mouth towards the closed position.

13. An assembly for applying liquid to an eye of a user, the assembly comprising:
a squeezable eye drop vial containing a liquid compatible with the eye therein;
a flexible rim member defining a mouth;
a flexible bowl member, integral with the flexible rim member; and
an elastomeric neck integral with an opening to the bowl, the elastomeric neck
further having an opening adapted to receive the fluid bearing container.
